(19) United States
(12) Patent Application Publication
(10) Pub. No.: US 2022/0226678 A1
(43) Pub. Date: Jul. 21, 2022

(54) MASK

(71) Applicant: Paul Moran, Neenah, WI (US)

(72) Inventor: Paul Moran, Neenah, WI (US)

(21) Appl. No.: 17/612,322

(22) PCT Filed: Mar. 12, 2021
(86) PCT No.: PCT/US2021/022175

§ 371 Date: Nov. 18, 2021

Related U.S. Application Data
(60) Provisional application No. 62/989,271, filed on Mar. 13, 2020.

(51) Int. Cl. A62B 23/02; A62B 18/10; A61L 9/20

(52) U.S. Cl. CPC A62B 23/02; A61L 2209/14; A61L 9/20; A62B 18/10

(57) ABSTRACT

A mask assembly includes a mask configured to cover a mouth and nasal passage of the user, a valve coupled to the mask and configured to direct air out of the mask assembly, and a strap coupled to the mask. The mask assembly additionally includes a module removably coupled to the mask and configured to direct air into the mask assembly. The module includes a filter, an ultraviolet chamber, and a light source.

### CROSS-REFERENCE TO RELATED APPLICATIONS

This application claims priority to U.S. Provisional Patent Application No. 62/989,271 filed on Mar. 13, 2020, the entire contents of which are incorporated herein by reference.

### BACKGROUND

Traditional masks are often designed to conceal a portion of a user's face, such as the mouth and nasal passage. When the user wears such masks in high risk environments (e.g., hospitals), the mask prevents the user from inhaling air containing bacteria and viruses.

### SUMMARY OF THE INVENTION

The present disclosure relates to a mask assembly, and more particularly to a UV light disinfectant mask assembly.

The disclosure provides, in one aspect, a mask assembly including a mask configured to cover a mouth and nasal passage of the user, a valve coupled to the mask and configured to direct air out of the mask assembly, a strap coupled to the mask, and a module removably coupled to the mask and configured to direct air into the mask assembly, wherein the module includes a filter, an ultraviolet chamber, and a light source.

The disclosure provides, in another aspect, a mask assembly including a mask configured to cover a mouth and nasal passage of a user, a strap coupled to the mask, a module removably coupled to the mask, and a filter assembly positioned within the module, the filter assembly including a first filter coupled to an external surface of the module, a second filter positioned within the module, a ultraviolet chamber, and a light emitting diode, wherein the light emitting diode and the ultraviolet chamber are operable to disinfect air flowing through the filter assembly.

The disclosure provides, in another aspect, a mask assembly including a mask configured to cover a mouth and nasal passage of the user, a valve coupled to the mask and configured to direct air out of the mask assembly, a strap assembly coupled to the mask, the strap assembly including a first strap configured to engage a first portion of the user's head, and a second strap configured to engage a second portion of the user's head, a first module removably coupled to the mask and configured to direct air into the mask assembly, the first module including a first filter assembly, and a second module removably coupled to the mask and configured to direct air into the mask assembly, the second module including a second filter assembly.

Other features and aspects of the disclosure will become apparent by consideration of the following detailed description and accompanying drawings.

### BRIEF DESCRIPTION OF THE DRAWINGS

FIG. 1 is a front perspective view of a mask assembly in accordance with an embodiment of the invention.

FIG. 2 is a front view of the mask assembly of FIG. 1.

FIG. 3 is a front view of the mask assembly of FIG. 1, illustrating a flow path of air flowing through the mask assembly.

FIG. 4 is a partially exploded view of the mask assembly of FIG. 1.

FIG. 5 is an exploded view of a module of the mask assembly of FIG. 1.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting.

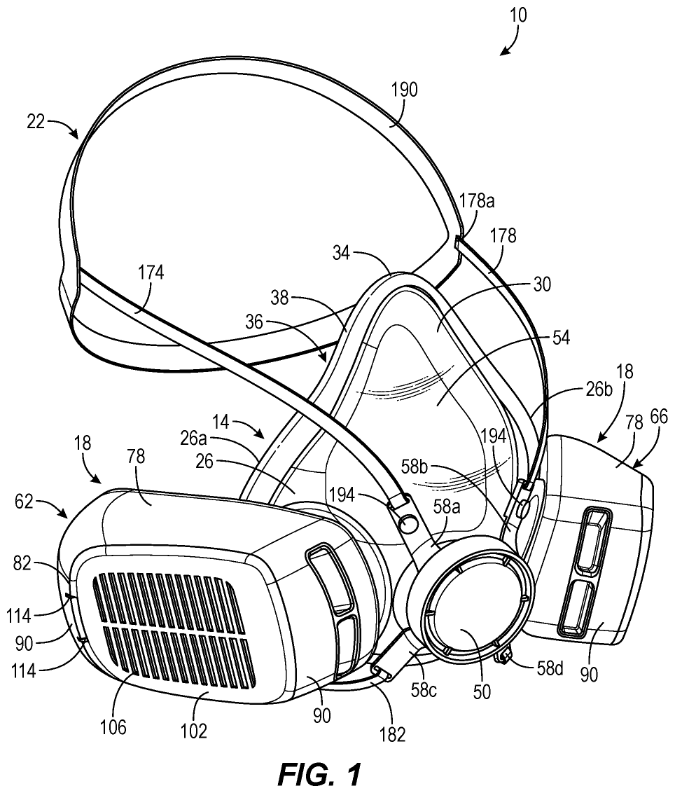

FIGS. 1-5 illustrate a UV light disinfectant mask assembly 10 according to one embodiment of the invention. The UV light disinfectant mask assembly 10 may be secured to a user's head, around a user's mouth, allowing the mask assembly 10 to destroy germs or viruses around the mouth and nasal passage. The mask assembly 10 includes a mask 14, a module assembly 18, and a strap assembly 22. When the mask assembly 10 is worn by the user, the mask 14 is preferably positioned over the user's mouth and nose, and the module assembly 18 is positioned on opposite sides of the mask 18, resting on the user's cheeks.

Referring to FIGS. 1-3, the illustrated mask 14 is shaped to fit over a user's mouth and nose. As illustrated, the mask 14 is teardrop shaped and includes a first, generally circular, portion 26 shaped to fit over a user's mouth and a second portion 30 shaped to fit over a user's nose. The particular shape of the mask 14 should not be considered limiting as other suitable shapes that fit over the user's mouth and nose are within the scope of the present disclosure. The second portion 30 is generally tapered and includes a rounded edge 34. In the illustrated embodiments, the mask 14 is constructed of a flexible and transparent plastic material. However, in some embodiments, the mask 14 may be constructed of a rigid material. An inner perimeter 36 of the mask 14 is lined with a cushion 38, which forms a seal against the user's face during use, thereby preventing air from unintentionally leaking out of the mask 14.

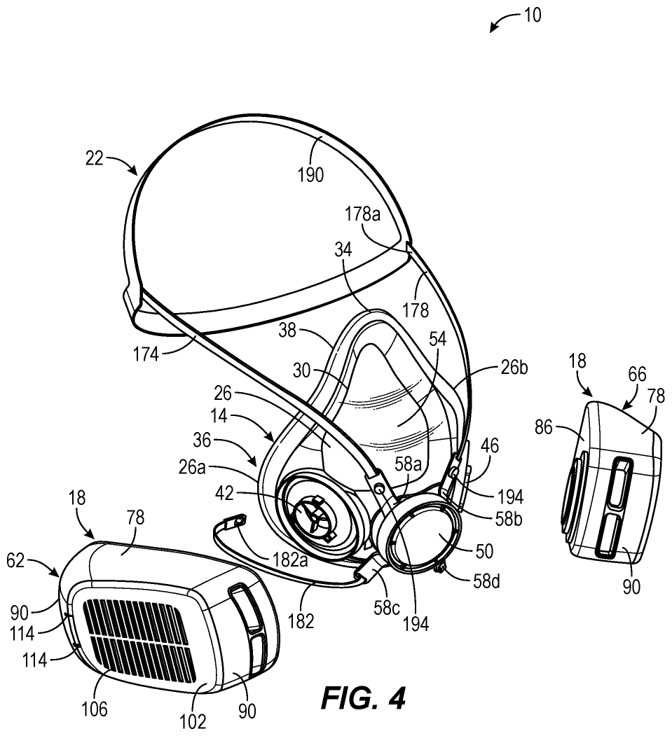

With reference to FIG. 4, the mask 14 further includes a first connector 42 and a second connector 46 positioned on opposite ends of the mask 14. The first connector 42 is positioned on a first end 26a of the first portion 26 of the mask 14, and the second connector 46 is positioned on a second end 26b of the first portion 26 of the mask 14. The first and second connectors 42, 46 are generally cylindrical and are shaped and sized to receive a portion of the module assembly 18 to couple the module assembly 18 to the mask 14.

With reference to FIGS. 1-4, the mask 14 includes a valve 50 coupled to a front surface 54 of the mask 14. The valve 50 is a one-way valve operable to discharge air exhaled from the user out of the mask 14, to the surrounding environment. The valve 50 is generally cylindrical and includes flanges 58a, 58b, 58c, 58d equidistantly spaced around a perimeter of the valve 50. More specifically, the valve 50 includes a first flange 58a, a second flange 58b, a third flange 58c, and a fourth flange 58d. Each of the flanges 58a, 58b, 58c, 58d are coupled to the strap assembly 22.

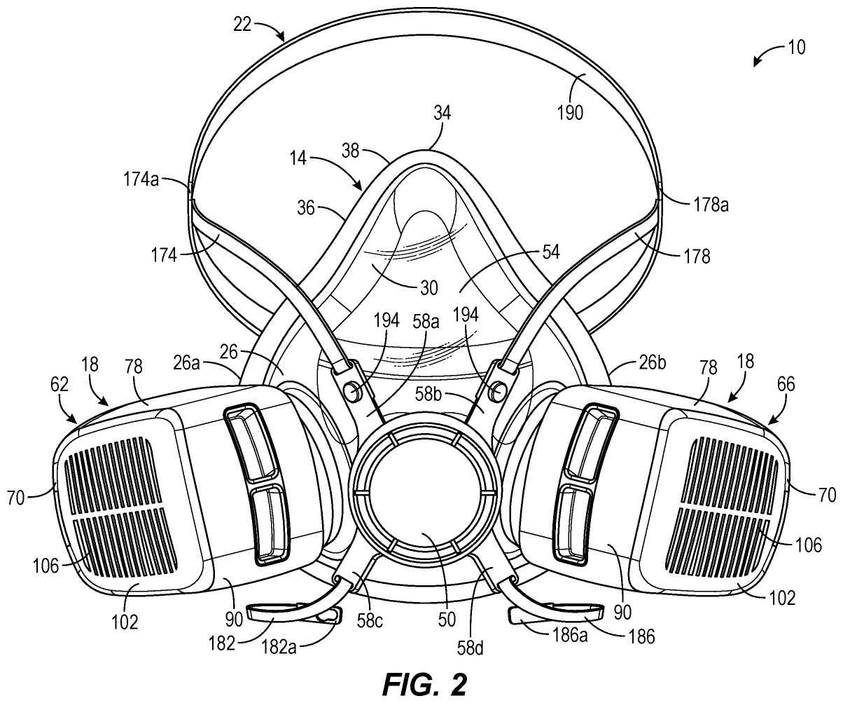

Referring to FIGS. 2-3 and 5, the module assembly 18 includes a first module 62 and a second module 66. Each of the modules 62, 66 includes a body 70 having a front wall 74, a back wall 78 opposite the front wall 74, a top wall 82, a bottom wall 86 opposite the top wall 82, and two side walls 90 the extend between the top wall 82 and the bottom wall 86. In the illustrated embodiment, the modules 62, 66 are generally rectangular and the side walls 90 are curved. In alternative embodiments, the modules 62, 66 may include different configurations. As will be described in further detail below, an internal chamber 94 of each of the modules 62, 66 houses a filter assembly 98.

The top wall 82 of each of the modules 62, 66 supports a door 102. The door 102 is generally rectangular and includes several elongate apertures 106. The door 102 is positioned on an external filter 110 of the filter assembly 98, which is positioned on the top wall 82 of the body 70. The apertures 106 extending through the door 102 allow for air to flow through the door 102 and into the external filter 110. In the illustrated embodiment, the door 102 is a sealed door having, for example, a gasket extending around an inner perimeter of the door 102 to inhibit fluid and dust ingress. The illustrated door 102 includes a latch 114. In order to access the external filter 110, the user may grasp (e.g., pinch) the latch 114, which disengages the latch 114 from a corresponding hinge 118 on the module 62, 66, thereby allowing the user to pivot the door 102 open about a pivot point 122.

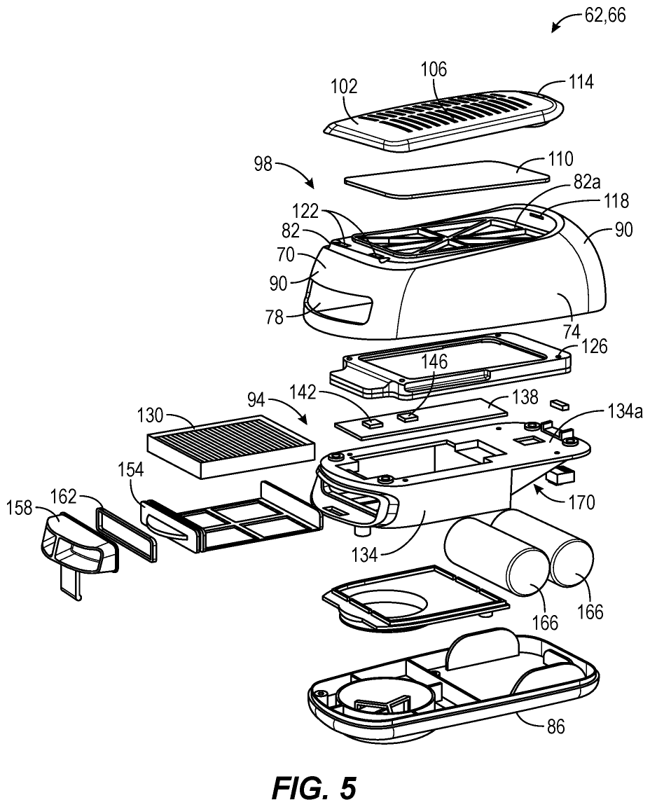

With reference to FIG. 5, the filter assembly 98 includes the external filter 110, an ultraviolet (UV) chamber 126, and an internal filter 130. The external filter 110 is positioned on an external surface 82a of the top wall 82 of the body 70, between the top wall 82 and the door 102. In the illustrated embodiments, the external filter 110 is composed of paper and is replaceable by a user after extended use. However, in some embodiments, the external filter 110 may be composed of different materials or may be permanently installed within the module 62, 66. The external filter 110 is configured to remove airborne dust particles and debris entering the module 62, 66 from the environment.

With continued reference to FIG. 5, the internal chamber 94 of the module 62, 66 includes a chassis 134. The UV chamber 126 is an elongate frame positioned between the top wall 82 and the chassis 134. More specifically, the UV chamber 126 is seated within the internal chamber 94 and abuts against an internal surface of the top wall 82. In the illustrated embodiments, the UV chamber 126 is generally rectangular and composed of aluminum. However, the UV chamber 126 may be composed of alternative materials and/or include different shapes and sizes.

A printed circuit board (PCB) 138 is positioned between the UV chamber 126 and the chassis 134, such that the PCB 138 is seated on an upper surface 134a of the chassis 134. The PCB 138 includes a first light source 142 and a second light source 146, such that each of the light sources 142, 146 include one or more light emitting diodes (LEDs). In particular, the first light source 142 is a first LED, and the second light source 146 is a second LED. In other embodiments, the first and second light sources 142, 146 may include any number or arrangement of LEDs, which may be mounted to one or more PCBs. The UV chamber 126 includes apertures shaped and sized to receive the first and second light sources 142, 146. Therefore, during operation of the mask assembly 10, the first and second light sources 142, 146 emit light through the UV chamber 126. The light sources 142, 146 emit light within the chamber 126, thereby forming a high intensity zone capable of killing airborne bacteria and viruses (e.g., biomass particles) found within the air passing through the module 62, 66.

The internal filter 130 is seated within the chassis 134. More specially, the chassis 134 includes a drawer 154 slidably received within the chassis 134. The drawer 154 is shaped and sized to receive the internal filter 130. When the internal filter 130 is seated within the drawer 154, the drawer 154 is positioned within the chassis 134, and a seal 158 and an O-ring 162 are secured to an edge of the drawer 154, thereby securing the internal filter 130 within the chassis 134. In the illustrated embodiments, the internal filter 130 is composed of paper and is replaceable by a user after extended use. However, in some embodiments, the internal filter 130 may be composed of different materials or may be permanently installed within the module 62, 66. The internal filter 130 is configured to remove airborne dust particles and debris entering the module 62, 66 from the environment.

The bottom wall 86 of each of the modules 62, 66 includes a circular port 166. The port 166 is shaped and sized to fit within the first and second connectors 42, 46 on the mask 14, thereby removably coupling the modules 62, 66 to the mask 14. The connectors 42, 46 engage the port 166 in a “snap-fit” configuration. In some embodiments, the modules 62, 66 may be removably coupled or permanently coupled to the mask 14 via alternative configurations.

With reference to FIG. 5, the mask assembly 10 includes a power source 166 positioned within the module assembly 62, 66 and electrically coupled to the filter assembly 98 (e.g., the first light source 142 and the second light source 146). In the illustrated embodiment, the power source 166 includes a single cell battery. More particularly, the power source 166 is a lithium-ion battery. However, in alternative embodiments, the battery 166 may include different chemistries. The power source 166 is positioned within a battery compartment 170 of the module 62, 66 so that the power source 166 can be removed. In the illustrated embodiment, the battery compartment 170 is positioned adjacent to (e.g., behind) the PCB 138. The battery compartment 170 includes battery terminals electrically coupled to the PCB 138 to provide power from the battery 166 to the light sources 142, 146. In some embodiments, the module 62, 66 may include a charging port (e.g., a USB port) electrically connected to the power source 166.

The battery 166 is insertable and removable from the battery compartment 170 by removing the bottom wall 86 of the module 62, 66, which may be pivotally coupled to the battery compartment 170. Alternatively, the bottom wall 86 may be coupled to the battery compartment 170 in other ways. The battery 166 can be removed for charging, and optionally replaced by a similar battery to allow for continued operation of the module 62, 66. In other embodiments, the battery 166 may not be removable from the body 70. In yet other embodiments, the battery 166 may be a single-use battery (e.g., an alkaline battery).

The module assembly 18 is operable remotely using any suitable communication scheme (e.g., wireless communication scheme, Bluetooth). In some embodiment, Bluetooth may be used to remotely control the module assembly 18 (e.g., turn the filter assembly 98 ON and OFF, thereby turning the first and second LEDs 142, 146 ON and OFF, respectively). Alternatively, in some embodiments, the mask assembly 10 includes a control panel for controlling operation of the module assembly 18. In such embodiments, the control panel includes an actuator (e.g., a button) to operate the module assembly 18. In still further embodiments, the module assembly 18 may include a light intensity control, which allows a user to increase or decrease the intensity of the light sources 142, 146.

With reference to FIGS. 1-2 and 4, the strap assembly 22 is removably coupled to the valve 50 and includes a first strap 174, a second strap 178, a third strap 182, a fourth strap 186, and a securing portion 190. The straps 174, 178, 182, 186 are removably coupled to the flanges 58a-58d on the valve 50 and extend away from the valve 50. The straps 174, 178, 182, 186 are removably coupled to the flanges 58a-58d via fasteners (e.g., snaps, pins, etc.) 194. However, in some embodiments, the straps 174, 178, 182, 186 may be permanently coupled to the valve 50. A distal end 174a of the first strap 174 and a distal end 178a of the second strap 178 are coupled to and integrally formed with the securing portion 190. The securing portion 190 is a circular strap shaped and sized to extend around an upper portion of a user's head, thereby securing the second portion 30 of the mask 14 to the user. The third strap 182 includes a distal, free end 182a configured to removably couple to a distal, free end 186a of the fourth strap 186. The third and fourth straps 182, 186 may extend (e.g., wrap) around a lower portion of a user's head, or neck, and the distal ends 182a, 186a may be coupled together, thereby securing the first portion 26 of the mask 14 to the user.

In order to secure the mask assembly 10 to the user, the user first positions the mask 14 against their face such that the first portion 26 covers the user's mouth and the second portion 30 covers the user's nose. The user positions the securing portion 190 of the strap assembly 22 around an upper portion of the user's head, and wraps the third and fourth straps 182, 186 around a lower portion of the user's head and secures the distal ends 182a, 186a of the third and fourth straps 182, 186 together.

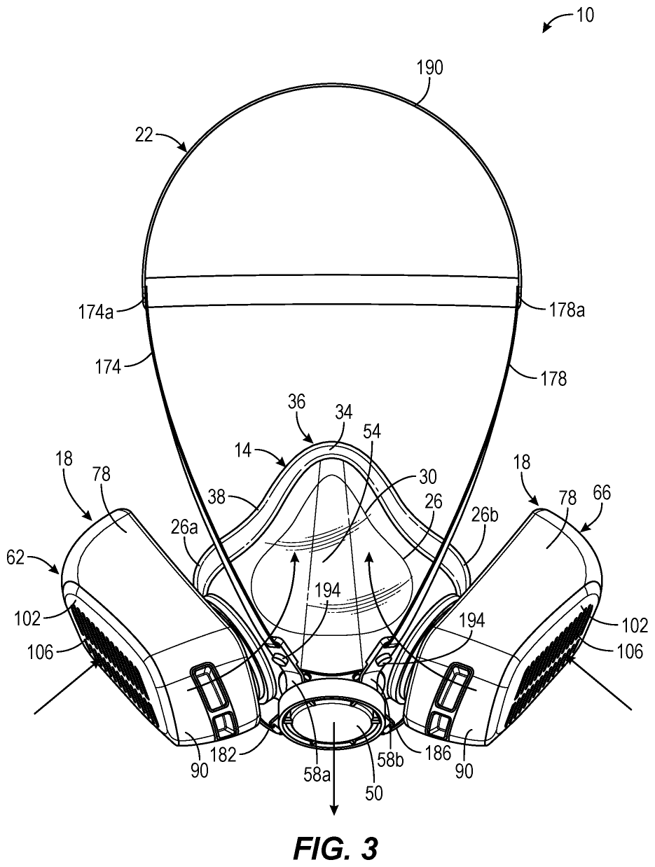

In order to operate the mask assembly 10, the user remotely turns the module assembly 18 ON via the communication scheme. Upon actuation of the module assembly 18, the first and second light sources 142, 146 emit light. With reference to FIG. 3, as the user breathes in, the air flows through the modules 62, 66. Specifically, the air flows through the top wall 82 and through external filter 110, the UV chamber 126, and the internal filter 130. The air exits the modules 62, 66 via the bottom wall 86 and then enters the mask 14 via the first and second connectors 42, 46. The external filter 110 and the internal filter 130 remove airborne dust particles, and the UV chamber 126 and the LEDs 142, 146 irradiate germs and bacteria, thereby sterilizing the air. The mask 14 acts as a secondary filter assembly and provides protection against UV light radiation from the UV chamber 126, enabling a user to safely wear the mask for an extended period of time (e.g., hours). As the user exhales, the air exits the mask 14 and flows through the one-way valve 50.

Various features of the invention are set forth in the following claims.

What is claimed is:

1. A mask assembly comprising:
a mask configured to cover a mouth and nasal passage of the user;
a valve coupled to the mask and configured to direct air out of the mask assembly;
a strap coupled to the mask; and
a module removably coupled to the mask and configured to direct air into the mask assembly, wherein the module includes a filter, an ultraviolet chamber, and a light source.

2. The mask assembly of claim 1, wherein the filter is a first filter coupled to an external surface of the module, and wherein the module includes a second filter positioned within the module.

3. The mask assembly of claim 1, wherein the light emitting diode and the ultraviolet chamber are operable to disinfect air flowing through the module.

4. The mask assembly of claim 1, wherein the strap is a first strap configured to engage a first portion of the user's head, and wherein the mask assembly further includes a second strap configured to engage a second portion of a user's head.

5. The mask assembly of claim 1, wherein the module includes a battery compartment configured to house a battery.

6. The mask assembly of claim 1, further comprising a wireless communication scheme configured to operate the light source.

7. The mask assembly of claim 1, wherein the valve is a one-way valve.

8. A mask assembly comprising:
a mask configured to cover a mouth and nasal passage of a user;
a strap coupled to the mask;
a module removably coupled to the mask; and
a filter assembly positioned within the module, the filter assembly including
a first filter coupled to an external surface of the module,
a second filter positioned within the module,
a ultraviolet chamber, and
a light emitting diode, wherein the light emitting diode and the ultraviolet chamber are operable to disinfect air flowing through the filter assembly.

9. The mask assembly of claim 8, wherein the module includes a printed control board, and wherein the light emitting diode is positioned on the printed control board.

10. The mask assembly of claim 8, wherein the light emitting diode is a first light emitting diode, and wherein the filter assembly further includes a second light emitting diode.

11. The mask assembly of claim 8, wherein the first filter and the second filter are replaceable.

12. The mask assembly of claim 8, wherein the ultraviolet chamber and the light emitting diode are positioned between the first filter and the second filter.

13. The mask assembly of claim 8, wherein the filter assembly includes a seal configured to secure the second filter within the module.

14. The mask assembly of claim 8, further comprising a valve coupled to the mask, wherein the module is configured to direct air into the mask assembly and the valve is configured to direct air out of the mask assembly.

15. A mask assembly comprising:
a mask configured to cover a mouth and nasal passage of the user;
a valve coupled to the mask and configured to direct air out of the mask assembly;
a strap assembly coupled to the mask, the strap assembly including a first strap configured to engage a first portion of the user's head, and a second strap configured to engage a second portion of the user's head;
a first module removably coupled to the mask and configured to direct air into the mask assembly, the first module including a first filter assembly; and
a second module removably coupled to the mask and configured to direct air into the mask assembly, the second module including a second filter assembly.

16. The mask assembly of claim 15, wherein the first module is removably to a first end of the mask, and the second module is removably coupled to a second end of the mask opposite the first end.

17. The mask assembly of claim 15, wherein the mask includes a first connector configured to couple to the first module, and a second connector configured to couple to the second module.

18. The mask assembly of claim 15, wherein the valve includes a plurality of flanges, wherein the first strap and the second strap are removably coupled to the flanges.

19. The mask assembly of claim 15, wherein the first filter assembly includes a first plurality of light sources, and the second filter assembly includes a second plurality of light sources.

20. The mask assembly of claim 19, wherein the first plurality of light sources are operable to disinfect air flowing through the first module, and the second plurality of light sources are operable to disinfect air flowing through the second module.
